(19) United States
(12) Patent Application Publication
(10) Pub. No.: US 2009/0182055 A1
(43) Pub. Date: Jul. 16, 2009

(54) FUNGICIDAL AGENT CONTAINING N-CHLOROTAURINE AND USE THEREOF

(76) Inventors: Waldemar Gottardi, INNSBRUCK (AT); Markus NAGL, Axams (AT); Andreas NEHER, INNSBRUCK (AT)

Correspondence Address: Frederic P. Zotos, 28 Old Coach Road, Cohasset, MA, 02025, US

(21) Appl. No.: 11/866,924

(22) Filed: Oct. 3, 2007

Related U.S. Application Data
(62) Division of application No. 10/380,414, filed on Feb. 2, 2004, now abandoned.

(30) Foreign Application Priority Data
Sep. 14, 2000 (DE) 100 45 868.8

(51) Int. Cl. A61K 31/185; A61P 17/00; A61P 31/10; A61P 15/00; A61P 1/00

(52) U.S. Cl. 514/578

(57) ABSTRACT

A fungicidal agent for treating infectious diseases caused by fungi contains N-chlorotaurine or one of its salts in aqueous solution, optionally with customary pharmaceutical additives.

This application is a divisional of patent application Ser. No. 10/380,414 filed Feb. 2, 2004 (abandoned), which is a US National Phase of International Application No. PCT/EP01/10497 filed Sep. 10, 2001, which claims the benefit of German priority application DE 100 45 868.8, filed Sep. 14, 2000.

### BACKGROUND OF THE INVENTION

The present invention relates to a fungicidal agent which is suitable for the treatment of the acute or chronic rhinosinusitis (ARS or CRS) and other diseases caused by fungal infection such as otitis, dermatitis, e.g., ulcus cruris; bronchitis; pneumonias, such as, for example, pneumocystis carinii; diseases of the sex organs caused by fungi such as colpitis, endometritis, or balanitis; diseases of the gastro-intestinal tract caused by fungi such as stomatitis, esophagitis, or enteritis; or urinary tract infections such as pyelonephritis, ureteritis, cystitis, ureteritis as well as the use of the agent.

In nasal mucus of healthy persons and patients suffering from ARS or CRS numerous fungal species have been demonstrated (Mayo Clin. Proc. 1999, 74, 877-884). For the treatment of ARS and CRS antibiotics (e.g., fusafungin), cortisone, mast cell degranulation inhibitors, and antihistamines have been used previously, in the case of polyposis cortisone in particular, also in connection with surgical interventions. In particular in the case of CRS the fungistatics used have proven themselves less effective.

Based on previous experience the treatment of fungal infections, in particular in the case of immunosupression, is less effective with the known fungistatics.

Surprisingly it was found that N-chlorotaurine (NCT), in particular in the form of its salts, such as, for example, sodium salt (NCT-Na), has an outstanding microbicidal effect in the treatment of diseases caused by fungal infections, such as, for example, ARS and CRS.

NCT-Na was synthesized for the first time in 1991 and has presented itself in the meantime as a versatile new disinfection agent in human medicine. This is based on the one hand on its wide-spectrum microbicidal effect, which has already been substantially documented with respect to bacteria and viruses in vitro.

In the case of rhinosinusitis various species of fungus were found in the nasal mucus, e.g., aspergillus, penicillum, fusarium, and candida. If (diluted 1:10) 1% NCT-Na is added to this nasal mucus, then the kill-off time in vitro is less than 30 minutes.

NCT-Na is tolerated outstandingly in vivo, which could be demonstrated in the eye, in the middle ear, in the outer ear, in skin ulcers, in the oral cavity, and the bladder.

Trials, e.g., with rhinosinusitis patients, showed that by treatment of the internal nasal surfaces with NCT-Na solution the symptoms of this disease such as pains, head pressure, loss of the sense of smell, increased secretion, and polyp formation have improved substantially and even freedom from complaints occurred. Treatments with traditional fungicidal agents have on the contrary previously led to no success.

The importance of the agent lies, among other things, in the fact that, in the case of the mucus tissue polyps occurring in rhinosinusitis, regression could be observed so that the previously successful standard therapy, namely operative removal, can be avoided by the use of the agent according to the invention.

### SUMMARY OF THE INVENTION

It is the objective of the present invention to provide a means which makes possible a successful therapy of diseases caused by fungal infection, among others ARS or CRS, under protective conditions and with a minimal risk for the patient.

This objective is realized according to the invention by a generic agent with the features of the characterizing clause of the main claim. The subordinate claims describe preferred developments of the invention.

The agent according to the invention for the treatment of diseases caused by fungal infection can be applied in the following way to the external and internal surfaces affected:
rinsing
inhalation
spraying
painting
orally (swallowing)

As customary additives, by way of example, the following substances, among others, can be added to the agent according to the invention:
cooking salt, for example for setting an isotonic solution
buffer substances
stabilizers
flavoring and scenting agents
preservatives

The concentration of N-chlorotaurine can vary within wide limits and, by way of example, in the range of 0.001 to 50%, preferably in the range of 0.1 to 10%, in particular in the range of 0.5 to 2%. In the practical application a concentration of ca. 1% has proven itself expedient, in particular in treatment by rinsing or spraying.

The percentages specified in this application relate to percentage by weight unless otherwise specified.

### DETAILED DESCRIPTION OF THE INVENTION

The invention will be explained in more detail in the following with the aid of the examples.

### Example 1

A 62-year-old man suffers from chronic rhinosinusitis with clear swelling of the mucus membranes in both maxillary sinuses with retention of secretion on the left, in ethmoid bone cells, sphenoidal sinus, and in the left frontal sinus. Labored nasal respiration. For 2 1/2 months increasing headaches at the back of the head, no improvement on antibiotic therapy.

The treatment with 1% NCT-Na took place over a total of 4 weeks, 3 times weekly, and included 12 treatments in all. Each time a 10-ml NCT-Na solution was applied by means of a nasal catheter (YAMIK) over a period of time of 12 minutes. Thereafter the rinsing solution was suctioned off.

Result of the treatment: Already after the second application complete remission of the pains. After 7 treatments strong decline in the formation of secretion, after 9 treatments clearly improved nasal respiration.

### Example 2

33-year-old patient suffered from chronic rhinosinusitis for ca. 1 year with swelling of the mucus membranes and increased secretion in the nasal cavity and all its adjacent cavities, strong polyposis and loss of the sense of smell.

The treatment with 1% NCT-Na took place over a total of 4 weeks, 3 times weekly, and included 12 treatments in all. Each time a 10-ml NCT-Na solution was applied by means of a nasal catheter (YAMIK) over a period of time of 12 minutes. Thereafter the rinsing solution was suctioned off.

Result of the treatment: Already after the sixth application a substantial restoration of the sense of smell. At the end of the treatments an unambiguous regression of the polyposis nasi was to be found.

### Example 3

A group of 10 patients who suffered from otitis externa with affliction of the auditory canal were treated. For treatment, 12 cm long strips (tampons by Rauscher) were placed into the external auditory canal. The test group (5 persons) was treated with strips which were saturated with 1-ml 1% NCT-Na, the control group (5 persons) with strips which were saturated with 1-ml otosporin (content per ml: 10,000 IU polymyxin B sulfate, 3.5 mg neomycin as neomycin sulfate, 10 mg hydrocortisone).

The treatment was done over 5 days. The strips were changed daily.

Result of the treatment: NCT-Na proved itself well tolerated. The period until healing was 3 days on average for the NCT-Na group, 5 days in the otosporin group.

### Example 4

A group of 25 patients who suffered from ulcus cruris were treated. The treatment was done with swabs which were saturated with an aqueous solution of 1% NCT-Na (test group, 14 patients) or 1% chloramine T (control group, 11 patients) and applied directly to the ulcers and left there. The treatment was done over 5-14 days. The swabs were changed twice daily.

Result of the treatment: In both groups the symptoms of inflammation could be made to disappear in 5 to 7 days. In the NCT-Na group significantly less pains occurred after application of the solution.

1-13. (canceled)

14. A method for the treatment of dermatitis in a mammal, comprising the step of administering to the mammal a therapeutically effective amount of N-chlorotaurine or a pharmaceutically acceptable salt thereof.

15. A method for the treatment of a venous ulcer in a mammal, comprising the step of administering to the mammal a therapeutically effective amount of N-chlorotaurine or a pharmaceutically acceptable salt thereof.

16. A method for the treatment of a sex organ disease in a mammal, comprising the step of administering to the mammal a therapeutically effective amount of N-chlorotaurine or a pharmaceutically acceptable salt thereof.

17. A method for the treatment of a gastrointestinal disease in a mammal, comprising the step of administering to the mammal a therapeutically effective amount of N-chlorotaurine or a pharmaceutically acceptable salt thereof.

18. The method of claim 16, wherein the disease is selected from the group consisting of: colpitis, endometritis and balanitis.

19. The method of claim 17, wherein the disease is selected from the group consisting of: stomatitis, esophitis and enteritis.

20. The method of any one of claims 14 to 17, wherein the concentration of N-chlorotaurine or a pharmaceutically acceptable salt thereof is in the range of 0.001 to 50% by weight.

21. The method of claim 20, wherein the concentration of N-chlorotaurine or a pharmaceutically acceptable salt thereof is in the range of 0.1 to 10% by weight.

22. The method of claim 20, wherein the concentration of N-chlorotaurine or a pharmaceutically acceptable salt thereof is in the range of 0.5 to 2% by weight.

23. The method of claim 20, wherein the concentration of N-chlorotaurine or a pharmaceutically acceptable salt thereof is about 1% by weight.

24. The method of any one of claims 14-17, wherein the pharmaceutically acceptable salt of N-chlorotaurine is an alkali salt.

25. The method of claim 24, wherein the composition comprises the sodium salt of N-chlorotaurine.
